(19) United States
(12) Patent Application Publication
(10) Pub. No.: US 2021/0038570 A1
(43) Pub. Date: Feb. 11, 2021

(54) COMPOSITIONS AND METHODS FOR TREATING CUTANEOUS FIBROSIS

(71) Applicant: Timber Pharmaceuticals, Inc., Woodcliff Lake, NJ (US)

(72) Inventor: Zachary Rome, Nyack, NY (US)

(73) Assignee: Timber Pharmaceuticals, Inc., Woodcliff Lake, NJ (US)

(21) Appl. No.: 16/978,185

(22) PCT Filed: Mar. 4, 2019
(86) PCT No.: PCT/US19/20545

§ 371 Date: Sep. 3, 2020

Related U.S. Application Data
(60) Provisional application No. 62/639,751, filed on Mar. 7, 2018.

(51) Int. Cl. A61K 31/422; A61P 17/02

(52) U.S. Cl. CPC A61K 31/422; A61K 9/0014; A61P 17/02

(57) ABSTRACT

The present invention relates to local or topical compositions containing a therapeutically effective amount of a selective endothelin-A (ET-A) receptor antagonist or inhibitor, preferably sitaxentan, and pharmaceutically acceptable salts thereof. The compositions are useful for treating a patient that has a condition involving cutaneous fibrosis or connective tissue disease.

### FIELD OF THE INVENTION

The present invention relates to local or topical compositions containing a therapeutically effective amount of a selective endothelin-A (ET-A) receptor antagonist or inhibitor, preferably sitaxentan (also known as sitaxsentan), and pharmaceutically acceptable salts thereof. The compositions are useful for treating a patient that has a condition involving cutaneous fibrosis or connective tissue disease.

### BACKGROUND OF THE INVENTION

Fibrosis is the formation of excess fibrous connective tissue in an organ or tissue. It is a common pathophysiological response to damage from a variety of stimuli including persistent infections, autoimmune reactions, allergic responses, chemical insults, radiation, and tissue injury. The repair process typically involves two distinct phases: a regenerative phase, in which injured cells are replaced by cells of the same type, leaving no lasting evidence of damage; and a phase known as fibroplasia or fibrosis, in which connective tissues replace normal parenchymal tissue. This process is initially beneficial; however, if it is not appropriately controlled an excess of extracellular matrix (ECM) components will permanently replace normal tissue as scar tissue and result in a pathogenic state. Fibrosis can result in many different organs and tissues and there are several different types of fibrotic diseases, e.g., idiopathic pulmonary fibrosis, liver cirrhosis, scleroderma or systemic sclerosis, progressive kidney disease, and cardiovascular fibrosis. In many instances, the effects of fibrosis and its complications can lead to significant morbidity, organ failure, and even death. See https://www.ncbi.nlm.nih.gov/pmc/articles/PMC2693329/.

Despite the fibrotic commonality, each type of fibrotic disorder has a distinct etiology and clinical manifestation, and the mechanism of fibrosis varies widely in each organ. For example, in fibrotic livers, the primary collagen producing cells arise not from fibroblasts, as it has been demonstrated for other organs, but from hepatic stellate cells, which in normal physiology have a quiescent phenotype and regulate vitamin A homeostasis. See https://www.ncbi.nlm.nih.gov/pubmed/18222966. Consequently, each fibrotic disorder will have different treatment objectives and approaches. For example, treating a fibrotic liver or cirrhosis of the liver will depend on the cause and extent of liver damage. People with cirrhosis from excessive use of alcohol may be able to minimize damage from stopping further alcohol use. However, people with primary biliary cirrhosis (also known as primary biliary cholangitis) often experience significantly delayed progression of cirrhosis from the drug ursodiol, a naturally occurring bile acid. It has been demonstrated that ursodiol has no beneficial effect on alcohol-induced cirrhosis. See https://www.mayoclinic.org/diseases-conditions/cirrhosis/diagnosis-treatment/drc-20351492 and https://www.ncbi.nlm.nih.gov/pubmed/12668982.

Some, but not all, examples of conditions of cutaneous fibrosis are scleroderma, cicatricial alopecia (otherwise known as scarring alopecia), and scars, e.g. hypertrophic or keloid scars. Conditions of cutaneous fibrosis have many unique and complicated features. For example, in scleroderma (one of the most notable conditions of cutaneous fibrosis), the pathogenesis is still unclear, and reports are often inconsistent; viral or bacterial infection, genetic factors, and autoimmune processes have all been proposed as the underlying cause. Relative to the other types of fibrosis, treatments for and research into conditions of cutaneous fibrosis are lacking and in great need. For example, there have been very few randomized and controlled therapeutic studies, and there are no FDA approved treatments for the cutaneous symptoms of scleroderma, the most serious condition of cutaneous fibrosis. See https://www.ncbi.nlm.nih.gov/pubmed/25672301.

Scleroderma can be either localized (i.e. only present in the skin) or systemic (i.e. other organs, in addition to the skin, are affected). The severity varies from individual to individual and can range from mild to life-threatening. Some, but not all, examples of the cutaneous symptoms include Raynaud's Phenomenon, swelling or puffiness in the hands, pain and stiffness in the joints, skin thickening, ulcerations, calcinosis, telangiectasia, dry skin, itchy skin, and sclerodactyly. Additionally, the hardening and tightening of the skin can be disfiguring and cause extreme psychosocial strain. Some examples of drugs that are used in an attempt to treat scleroderma are calcium channel blockers, phosphodiesterase inhibitors, prostacyclin analogues, steroids, and immunosuppressants. These treatments, however, are often ineffective and/or have serious side effects. Additionally, people with scleroderma experience a significantly lower quality of life and scleroderma places a considerable economic burden on health care systems and society as a whole. See https://www.ncbi.nlm.nih.gov/pubmed/28899803.

The endothelins (ET-1, ET-2, and ET-3) constitute a family of 21 amino acid peptides that act on two distinct high-affinity receptor subtypes, endothelin-A (ET-A) and endothelin-B (ET-B). Of these three peptides, ET-1 has been the most studied and is believed to be the most representative peptide of the axis. It can be induced in endothelial cells by many factors including mechanical stimulation, various hormones, and pro-inflammatory cytokines. ET-1 stimulates cardiac contraction and the growth of cardiac myocytes, regulates the release of vasoactive substances (it is a potent vasoconstrictor), stimulates smooth muscle mitogenesis, and may control inflammatory responses by promoting the adhesion and migration of neutrophils and by stimulating the production of pro-inflammatory cytokines. It has also been implicated in cancer progression, regulating the proliferation and migration of tumor cells and acting as a pro-angiogenic factor and inducer of stromal reaction. See https://www.ncbi.nlm.nih.gov/pubmed/27266371. Given their broad activity, therapeutically controlling the endothelins has been an area of interest for potential treatments for many different pathological conditions. Bosentan, a dual (i.e. a non-selective) ET-A/ET-B receptor antagonist, was developed for and is now FDA approved (in a tablet form) to treat pulmonary arterial hypertension (PAH). It is sometimes used “off-label” in the treatment armamentarium for scleroderma; however, it is often ineffective and associated with significant side effects. Notably, the FDA, after careful review, did not approve it for use in scleroderma. See https://www.ncbi.nlm.nih.gov/pmc/articles/PMC4474386/.

There are currently no FDA approved treatments for many conditions of cutaneous fibrosis, the most notable being scleroderma. Therefore, doctors must use unproven and experimental methods to try and control the condition. While oral treatment with bosentan is sometimes used in scleroderma, its effect is often modest and side effects limit utility. For example, it has only been shown to help prevent the emergence of new digital ulcers in scleroderma and has no effect on the healing of existing ulcers. Liver enzyme abnormalities are common, affecting about 10% of patients and resulting in the cessation of treatment in about 5%. Other common adverse effects include edema, fluid retention, anemia, and gastrointestinal effects. See https://www.ncbi.nlm.nih.gov/pmc/articles/PMC4474386/ and http://ntag.nhs.uk/docs/app/Bosentan%20for%20digital%20ulcers%20-%2ONETAG%20appraisal%20report%20%28Apr10%29.pdf#search=%22bosentan%2.

The shortcomings of bosentan in scleroderma represent the disappointment of endothelin antagonists as potential treatments across a range of conditions. Upon its discovery, there was great enthusiasm for targeting the endothelin axis as a mechanism for treating many different conditions. Within five years of the discovery of the endothelin axis, orally available endothelin receptor antagonists became available and their effects were evaluated in clinical trials for cardiovascular diseases, heart failure, pulmonary arterial hypertension, resistant arterial hypertension, stroke, subarachnoid hemorrhage, kidney diseases, and various cancers. Aside from treating pulmonary arterial hypertension, the results of most clinical trials for other indications were either neutral or negative, leading to the discontinuation of endothelin-receptor antagonist programs in many pharmaceutical companies. See https://www.ncbi.nlm.nih.gov/pubmed/27266371. Therapeutically controlling the endothelin axis can be a complicated, nuanced, and challenging task.

Current research suggests that there is either no benefit to selective antagonism of ET-A vs. ET-B or that dual antagonism is preferable. For example, in PAH, both ET-A-selective and dual ET-A/ET-B antagonists have been approved by FDA, and yet a close analysis of their clinical outcomes revealed that it was not possible to identify a clinically relevant advantage for one class of drug over the other. It was, however, observed that patients on ET-A-selective drugs experienced more significant adverse events, particularly fluid retention. This observation was not unique. Among other examples, Phase 3 clinical trials for two different ET-A-selective antagonists (one for diabetic neuropathy and the other for cancer) led to early study termination due to water retention or increased mortality. See https://www.ncbi.nlm.nih.gov/pubmed/27266371.

Research efforts were looking for selective endothelin inhibitors, particularly compounds selective for inhibiting ET-A versus ET-B. Sitaxentan, a selective ET-A antagonist, was developed as an oral tablet for treating pulmonary arterial hypertension (PAH). Sitaxanten gained regulatory approval in Europe, but was voluntarily withdrawn from the market within five years based on emerging safety concerns, particularly those associated with liver toxicity. Consequently, sitaxsentan never gained FDA approval in the United States. In their 2016 report titled “Endothelin-receptor antagonists beyond pulmonary arterial hypertension: cancer and fibrosis”, Aubert and Juillerat-Jeanneret state that in fibrosis related disorders, human clinical trials have clearly noted a deleterious effect, in particular, fluid retention, of blocking endothelin receptors.

As a further example, in an in vitro study using human dermal fibroblasts to examine the effects of endothelin-1 on fibroblast matrix gene expression and connective tissue remodeling, Shi-wen et al. concluded that inhibiting both ET-A and ET-B was necessary in order to prevent the biosynthesis of collagen. See https://www.ncbi.nlm.nih.gov/pubmed/11231316. Adding another layer of complexity, it has been demonstrated that at high concentrations, ET-A-selective antagonists may themselves display dual antagonistic properties for ET-A/ET-B. In view of these prior art teachings, it was therefore surprising that we discovered that sitaxentan was superior to bosentan in several important mechanisms of cutaneous fibrosis, including the production of collagen. It was also surprising to discover that sitaxsentan was significantly less cytotoxic to human skin cells than bosentan. Additionally, we have discovered a means of treating conditions of cutaneous fibrosis with substantially lower doses of endothelin antagonists than previously used. For example, when bosentan is used in scleroderma, oral dosing often reaches 250 mg/day, resulting in unwanted systemic side effects. In contrast, the local or topical compositions of the present invention can provide a benefit with plasma levels that are significantly less than those obtained from oral dosing of an ET-A inhibitor. Further, the novel approach of treating conditions of cutaneous fibrosis through the local or topical application of the active ingredient provides a means of avoiding the well-known and significant systemic side effects that have prevented the previous utility of these compounds.

It is apparent from the foregoing that there is an ongoing need for developing safe and effective treatments for conditions of cutaneous fibrosis. Therapeutically controlling the endothelins may offer important treatment opportunities but attempts thus far have been unsuccessful. It has been surprisingly found in the present invention that the local or topical application of a selective ET-A receptor antagonist may be safely administered to treat conditions of cutaneous fibrosis or connective tissue disorders.

It has surprisingly been found in the present invention that the selective endothelin-A inhibitor, sitaxentan, can be safely and effectively administered locally or topically to treat and provide relief for patients from conditions involving cutaneous fibrosis or connective tissue disorders.

### SUMMARY OF THE INVENTION

The present invention relates to methods of use and local or topical compositions for the local or topical application of selective ET-A receptor antagonists or inhibitors for the treatment of cutaneous fibrosis or connective tissue disease.

The present invention is based on the surprising discovery that sitaxentan, a highly selective ET-A receptor antagonist, was significantly more effective than both a vehicle control and than bosentan, a non-selective ET-A/ET-B receptor antagonist, at reducing collagen production, reducing viability, inducing apoptosis, and reducing fibroblast migration in human dermal fibroblasts induced with transforming growth factor beta 1 (TGF-β1) to stimulate a pro-fibrotic phenotype.

### BRIEF DESCRIPTION OF THE DRAWINGS

FIG. 1 shows scratch assay experimental results for male normal human dermal fibroblasts (NHDFs) that were exposed to 50 ng/mL of transforming growth factor-β1 (TGF-β1) for 24 hours, prior to treatments comparing sitaxentan (SIT, 100 μM), against bosentan (BOS, 100 μM as a comparator compound) and vehicle control (VC). Statistical significance is indicated as follows: *p<0.05, n=6. One-Way ANOVA using Dunnett's post-hoc analysis.

FIG. 2 shows scratch assay experimental results for female normal human dermal fibroblasts (NHDFs) that were exposed to 50 ng/mL transforming growth factor-β1 (TGF-β1) for 48 hours, prior to treatments comparing sitaxentan (SIT, 100 μM), against bosentan (BOS, 100 μM as a comparator compound) and vehicle control (VC). Statistical significance is indicated as follows: *p<0.05 to control, #p<0.05 between experimental groups, n=3. One-Way ANOVA using Tukey's honest significant difference (HSD) post-hoc analysis.

FIG. 3 shows experimental results on collagen production for male normal human dermal fibroblasts (NHDFs) that were treated for 48 hours with vehicle (VC), sitaxentan (SIT, 100 μM), or bosentan (BOS, 100 μM) in the presence of 50 ng/mL of transforming growth factor-β1 (TGF-β1). Additionally, FIG. 3 includes a comparison of these groups against the collagen content for male normal human dermal fibroblasts that were not stimulated with TGF-β1. Statistical significance is indicated as follows: *p<0.05 to vehicle control, #p<0.05 between experimental groups, n=6. One-Way ANOVA using Tukey's honest significant difference (HSD) post-hoc analysis.

FIG. 4 shows experimental results for male normal human dermal fibroblasts (NHDFs) which were stimulated with 50 ng/mL transforming growth factor-β1 (TGF-β1) for 48 hours. Viability was measured comparing sitaxentan (SIT, 100 μM), against bosentan (BOS, 100 μM as a comparator compound) and vehicle control (VC), and reported as relative fluorescence units (RFUs) on the y-axis. Statistical significance is indicated as follows: *p<0.05 to control, #p<0.05 between experimental groups, n=6. One-Way ANOVA using Tukey's honest significant difference (HSD) post-hoc analysis.

FIG. 5 shows experimental results for male normal human dermal fibroblasts (NHDFs) which were stimulated with 50 ng/mL transforming growth factor-β1 (TGF-β1) for 48 hours. Cytotoxicity was measured comparing sitaxentan (SIT, 100 μM), against bosentan (BOS, 100 μM as a comparator compound) and vehicle control (VC), and reported as relative fluorescence units (RFUs) on the y-axis. Statistical significance is indicated as follows: #p<0.05 between experimental groups, n=6. One-Way ANOVA using Tukey's honest significant difference (HSD) post-hoc analysis.

FIG. 6 shows experimental results for male normal human dermal fibroblasts (NHDFs) which were stimulated with 50 ng/mL transforming growth factor-β1 (TGF-β1) for 48 hours. Apoptosis was measured comparing sitaxentan (SIT, 100 μM), against bosentan (BOS, 100 μM as a comparator compound) and vehicle control (VC), and reported as relative light units (RLUs) on the y-axis. Statistical significance is indicated as follows: *p<0.05 to control, #p<0.05 between experimental groups, n=6. One-Way ANOVA using Tukey's honest significant difference (HSD) post-hoc analysis.

### DETAILED DESCRIPTION OF THE INVENTION

The present invention relates to a method for treating cutaneous fibrosis or a connective tissue disease, comprising locally or topically applying a therapeutically effective amount of a selective endothelin-A (ET-A) receptor antagonist or inhibitor to a mammal in need thereof.

In another aspect, the present invention relates to a method for treating cutaneous fibrosis, comprising locally or topically applying a therapeutically effective amount of a selective endothelin-A (ET-A) receptor antagonist or inhibitor to a mammal in need thereof.

In another aspect, the present invention relates to a method for treating a connective tissue disease, comprising locally or topically applying a therapeutically effective amount of a selective endothelin-A (ET-A) receptor antagonist or inhibitor to a mammal in need thereof.

In another aspect, the present invention relates to a method wherein the selective endothelin-A (ET-A) receptor antagonist or inhibitor has a selectivity of at least two-fold over endothelin-B (ET-B).

In another aspect, the present invention relates to a method wherein the selective endothelin-A (ET-A) receptor antagonist or inhibitor has a selectivity of at least five-fold over endothelin-B (ET-B).

In another aspect, the present invention relates to a method wherein the selective endothelin-A (ET-A) receptor antagonist or inhibitor has a selectivity of at least ten-fold over endothelin-B (ET-B).

In another aspect, the present invention relates to a method wherein the selective endothelin-A (ET-A) receptor antagonist or inhibitor has a selectivity of at least 100-fold over endothelin-B (ET-B).

In another aspect, the present invention relates to a method wherein the selective endothelin-A (ET-A) receptor antagonist or inhibitor has a selectivity of at least 1000-fold over endothelin-B (ET-B).

In another aspect, the present invention relates to a method wherein the selective endothelin-A (ET-A) receptor antagonist or inhibitor has a selectivity of at least 5000-fold over endothelin-B (ET-B).

In another aspect, the present invention relates to a method wherein the selective endothelin-A antagonist or inhibitor is sitaxentan or a pharmaceutically acceptable salt thereof.

In another aspect, the present invention relates to a method wherein the mammal is a human patient.

In another aspect, the present invention relates to a method wherein the cutaneous fibrosis or connective tissue disorder is selected from scleroderma, systemic sclerosis, localized scleroderma, diffuse systemic sclerosis, limited systemic sclerosis, Raynaud's phenomenon, Peyronie's disease, sclerodactyly, cutaneous ulcers, morphea, en coup de sabre, cicatricial alopecia, scarring alopecia (including, but not limited to, lichen planopilaris, frontal fibrosing alopecia, central centrifugal cicatricial alopecia, folliculitis decalvens, discoid lupus erythematous, and dissecting cellulitis), rheumatoid arthritis, lupus, lichen sclerosis, keloid scars, hypertrophic scars, burn scars, and combinations thereof.

In another aspect, the present invention relates to a method wherein the pharmaceutically acceptable salt is selected from an alkali metal salt, an alkaline earth metal salt, and an ammonium salt.

In another aspect, the present invention relates to a method wherein the alkali metal salt is selected from lithium, sodium, and potassium.

In another aspect, the present invention relates to a method wherein the alkali metal salt is sodium.

In another aspect, the present invention relates to a method wherein the pharmaceutically acceptable salt is sitaxentan sodium.

In another aspect, the present invention relates to a method wherein the selective endothelin-A (ET-A) receptor antagonist or inhibitor is applied at least one daily.

In another aspect, the present invention relates to a method wherein the selective endothelin-A (ET-A) receptor antagonist or inhibitor is applied at least twice daily.

In another aspect, the present invention relates to a method wherein the selective endothelin-A (ET-A) receptor antagonist or inhibitor is applied at least once weekly.

In another aspect, the present invention relates to a method wherein the selective endothelin-A (ET-A) receptor antagonist or inhibitor is applied at least twice weekly.

In another aspect, the present invention relates to a method wherein the selective endothelin-A (ET-A) receptor antagonist or inhibitor is applied at least once daily until the cutaneous fibrosis or connective tissue disease is treated.

In another aspect, the present invention relates to a method wherein the selective endothelin-A (ET-A) receptor antagonist or inhibitor is applied from a pharmaceutically acceptable composition.

In another aspect, the present invention relates to a method for treating cutaneous fibrosis or a connective tissue disease, comprising locally or topically applying a pharmaceutically acceptable composition comprising a therapeutically effective amount of a selective endothelin-A (ET-A) receptor antagonist or inhibitor to a mammal in need thereof.

In another aspect, the present invention relates to the use of a selective endothelin-A (ET-A) receptor antagonist or inhibitor in the manufacture of a medicament for local or topical delivery of a therapeutically effective amount of the selective endothelin-A (ET-A) receptor antagonist or inhibitor for treating cutaneous fibrosis or a connective tissue disease in a mammal in need thereof.

In another aspect, the present invention relates to a composition for local or topical delivery comprising a therapeutically effective amount of a selective endothelin-A (ET-A) receptor antagonist or inhibitor and a pharmaceutically acceptable carrier.

In another aspect, the present invention relates to a composition wherein the selective endothelin-A (ET-A) receptor antagonist or inhibitor has a selectivity of at least two-fold over endothelin-B (ET-B).

In another aspect, the present invention relates to a composition wherein the selective endothelin-A antagonist or inhibitor is sitaxentan or a pharmaceutically acceptable salt thereof.

In another aspect, the present invention relates to a composition wherein the pharmaceutically acceptable salt is sitaxentan sodium.

In another aspect, the present invention relates to a composition for administration to a mammal.

In another aspect, the present invention relates to a composition wherein said mammal is a human patient.

In another aspect, the present invention relates to a composition in the form of a unit dosage composition.

In another aspect, the present invention relates to a unit dosage composition comprising about 0.01 to about 1000 mg of sitaxentan or a pharmaceutically acceptable salt thereof, based on the weight of the sitaxentan active.

In another aspect, the present invention relates to a unit dosage composition comprising from about 0.001% to about 25% by weight sitaxentan or a pharmaceutically salt thereof, based on the weight of the sitaxentan active.

In another aspect, the present invention relates to a unit dosage composition comprising from about 0.01% to about 10% by weight sitaxentan or a pharmaceutically salt thereof, based on the weight of the sitaxentan active.

In another aspect, the present invention relates to a unit dosage composition comprising from about 0.1% to about 5% by weight sitaxentan or a pharmaceutically salt thereof, based on the weight of the sitaxentan active.

In another aspect, the present invention relates to a unit dosage composition comprising from about 0.2% to about 3% by weight sitaxentan or a pharmaceutically salt thereof, based on the weight of the sitaxentan active.

In another aspect, the present invention relates to a unit dosage composition demonstrating at least one of the following pharmacokinetic parameters selected from a Cₘₐₓ less than about 13 μg/ml, or a Cₘₐₓ less than about 7 μg/ml, or an AUC (area under the curve) less than about 40 μg hr/ml.

In another aspect, the present invention relates to a method for preparing a composition according to the present invention.

These and other aspects of the present invention will become apparent from the disclosure herein.

Definitions

As used herein, the following terms and abbreviations have the indicated meanings unless expressly stated to the contrary.

The term “selective” with respect to ET-A antagonist or inhibitor means an ET-A inhibitor which preferentially inhibits ET-A versus ET-B. The selectively for ET-A versus ET-B should be at least two-fold, preferably at least five-fold, more preferably at least ten-fold, more preferably at least 100-fold, more preferably at least 1000-fold, and most preferably at least 5000-fold. Such selectivity can be important for providing the therapeutic benefits of the present invention. A rationale for this selectively, compared to that for a non-selective inhibitor such as bosentan, is negligible inhibition of the beneficial effects of ET-B stimulation, such as nitric oxide production and clearance of endothelin from circulation.

The term “pharmaceutically acceptable” is used herein with respect to the compositions, in other words the formulations, of the present invention, and also with respect to the salts of sitaxentan, i.e. pharmaceutically acceptable salts. The pharmaceutical compositions of the present invention comprise a therapeutically effective amount of sitaxentan and a pharmaceutically acceptable carrier. These carriers can contain a wide range of excipients. Pharmaceutically acceptable carriers are those conventionally known carriers having acceptable safety profiles. The compositions are made using common formulation techniques. See, for example, Remington's Pharmaceutical Sciences, 17th edition, edited by Alfonso R. Gennaro, Mack Publishing Company, Easton, Pa., 17th edition, 1985. Regarding pharmaceutically acceptable salts, these are described below.

The term “subject” means a human patient or animal in need of treatment or intervention for cutaneous fibrosis or connective tissue disorders.

The term “therapeutically effective” means an amount of sitaxentan needed to provide a meaningful or demonstrable benefit, as understood by medical practitioners, to a subject, such as a human patient or animal, in need of treatment. Conditions, intended to be treated include, for example, cutaneous fibrosis and connective tissue disease. For example, a meaningful or demonstrable benefit can be assessed or quantified using various clinical parameters. The demonstration of a benefit can also include those provided by models, including but not limited to in vitro models, in vivo models, and animal models. An example of such a model is the Human Procollagen Type I C-peptide (PIP) assay. This model is designed to detect and quantify human procollagen in human serum, plasma, cell culture supernatants, cell lysate, and tissue homogenates in a variety of experimental states via AlphaLISA® technology. An example of an animal model which can be employed is the bleomycin induced skin fibrosis model. See, https://www.ncbi.nlm.nih.gov/pubmed/24706279.

The term “topical” as used herein with respect to pharmaceutical compositions means a composition that is applied to the skin or mucosal membrane of a subject, such as a human patient. A topical pharmaceutical composition is intended to have an effect at the site of application, i.e. in the tissue beneath the site of application, and does not result in significant drug concentrations in the blood and other tissues. Topical pharmaceutical compositions are in contrast to “transdermal” or “transmucosal” pharmaceutical compositions, which are absorbed through the skin or mucosal membranes and are intended to have a systemic effect in areas of the body away from the site of application. See, http://corporatepharmacy.ca/health-news/topical-vs-transdermal-meds, (2016).

Furthermore, the U.S. Food & Drug Administration has provided a standard for all routes of administration for drugs, i.e. “Route of Administration”. The following definitions are provided by the FDA for topical, transdermal, and transmucosal routes of drug administration.

| | | | | NCI* |
|---|---|---|---|---|
| | | SHORT | FDA | CONCEPT |
| NAME | DEFINITION | NAME | CODE | ID |
| TOPICAL | Administration | TOPIC | 011 | C38304 |
| | to a particular | | | |
| | spot on the | | | |
| | outer surface of | | | |
| | the body. | | | |
| TRANSDERMAL | Administration | T- | 358 | C38305 |
| | through the | DERMAL | | |
| | dermal layer of | | | |
| | the skin to the | | | |
| | systemic | | | |
| | circulation | | | |
| | by diffusion. | | | |
| TRANSMUCOSAL | Administration | T- | 122 | C38283 |
| | across the | MUCOS | | |
| | mucosa. | | | |

*National Cancer Institute
See, https://www.fda.gov/Drugs/DevelopmentApprovalProcess/FormsSubmissionRequirements/ElectronicSubmissions/DataStandardsManualmonographs/ucm071667.htm.

The term “local” as used herein with respect to pharmaceutical compositions means a route of administration of a composition in which the pharmacodynamic effect is generally contained around the application location and does not result in significant or rapid concentrations in the blood or other tissues. In addition to topical compositions, as defined above, some, but not all, examples of other local routes of administration can include subcutaneous injection and intradermal injection.

The terms “treat,” “treating” or “treatment,” as used herein, include alleviating, abating or ameliorating the condition, e.g. cutaneous fibrosis or connective tissue disease, or preventing or reducing the risk of contracting the condition or exhibiting the symptoms of the condition, ameliorating or preventing the underlying causes of the symptoms, inhibiting the condition, arresting the development of the condition, relieving the condition, causing regression of the condition, or stopping the symptoms of the condition, either prophylactically and/or therapeutically.

The methods of treatment using sitaxentan or a pharmaceutically acceptable salt thereof or the pharmaceutical compositions of the present invention, in various embodiments also include the use of sitaxentan or a pharmaceutically acceptable salt thereof in the manufacture of a medicament for the desired treatment, such as cutaneous fibrosis and connective tissue diseases.

“ET-A” is an abbreviation for endothelin-A.

“ET-B” is an abbreviation for endothelin-B.

“TGF-β1” is an abbreviation for transforming growth factor-β1.

“NHDF” is an abbreviation for normal human dermal fibroblasts.

Sitaxentan

The present invention utilizes a therapeutically effective amount of a selective endothelin-A (ET-A) receptor antagonist or inhibitor such as sitaxentan or a pharmaceutically acceptable salt thereof, and also a pharmaceutically acceptable carrier for providing local or topical compositions for treating conditions such as cutaneous fibrosis and connective tissue disorders.

Sitaxentan, also known as sitaxsentan, corresponds to the CAS Registry Number 184036-34-8 and the IUPAC name N-(4-Chloro-3-methyl-5-isoxazolyl)-2-[(2-methyl-4,5-methylenedioxyphenyl)-acetyl]thiophene-3-sulfonamide, and also the code name TBC-11251. Sitaxentan sodium salt, the form of the drug developed for human use, has the CAS Registry Number 210421-64-0. Sitaxentan was developed as an oral tablet for the treatment of pulmonary arterial hypertension (PAH) and was marketed as Thelin® by Encysive Pharmaceuticals until purchased by Pfizer in February 2008. In 2010, Pfizer voluntarily removed sitaxsentan from the market due to emerging safety concerns. http://press.pfizer.com/press-release/pfizer-stops-clinical-trials-thelin-and-initiates-voluntary-product-withdrawal-interes.

The chemical structure for sitaxentan is shown immediately below.

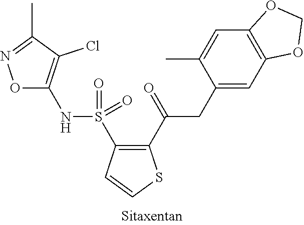 [SMILES: Cc1cc2c(cc1CC(=O)c1sccc1S(=O)(=O)Nc1onc(C)c1Cl)OCO2]

Sitaxentan has the chemical formula C₁₈H₁₅CIN₂O₂S₂ and a molar mass of 454.906 g/mol. The following pharmacokinetic data is reported:

Oral Bioavailability: 70 to 100%

Protein binding: >99%

Metabolism: hepatic (CYP2C9- and CYP3A4-mediated)

Biological half-life: 10 hours

Excretion: renal (50 to 60%), fecal (40 to 50%)

Sitaxentan is described as a small molecule that blocks or inhibits the action of endothelin (ET) on the endothelin-A (ET-A) receptor selectively. This selectivity is reported to be by a factor of 6000 compared to endothelin-B-(ET-B). See, Girgis, R E; Frost, A E; Hill, N S; Horn, E M; Langleben, D; McLaughlin, V V; Oudiz, R J; Robbins, I M; et al. (2007). “Selective endothelin-A receptor antagonism with sitaxsentan for pulmonary arterial hypertension associated with connective tissue disease”. Annals of the rheumatic diseases. 66 (11): 1467-72. doi:10.1136/ard.2007.069609. PMC 2111639 Freely accessible. PMID 17472992. Such selectivity can be important for providing the therapeutic benefits of the present invention.

Pharmaceutically acceptable salts of sitaxentan are useful for the methods and compositions of the present invention. As used herein, “pharmaceutically acceptable salts” refer to derivatives of sitaxentan modified by making salts thereof. Examples of pharmaceutically acceptable salts include, but are not limited to, alkali metal salts, alkaline earth metal salts, and ammonium salts. Examples of alkali metal salts include lithium, sodium, and potassium salts. Examples of alkaline earth metal salts include calcium and magnesium salts. The ammonium salt, NH4⁺. itself can be prepared, as well as various monoalkyl, dialkyl, trialkyl, and tetraalkyl ammonium salts. Also, one or more of the alkyl groups of such ammonium salts can be further substituted with groups such as hydroxy groups, to provide an ammonium salt of an alkanol amine. Ammonium salts derived from diamines such as 1,2-diaminoethane are contemplated herein. The sodium salt of sitaxentan, also called sitaxentan sodium, is useful herein. Lists of suitable salts are found in Remington's Pharmaceutical Sciences, 18th ed. (Mack Publishing Company, 1990).

The pharmaceutically acceptable salts of sitaxentan can be prepared from the parent compound by conventional chemical methods. Generally, such salts can be prepared by reacting the free acid form of the compound with a stoichiometric amount of the appropriate base in water or in an organic solvent, or in a mixture of the two; generally, non-aqueous media like ether, ethyl acetate, ethanol, isopropanol, or acetonitrile are preferred.

Dosages

In one aspect, the present invention comprises a therapeutically effective amount of sitaxentan or a pharmaceutically acceptable salt thereof and a pharmaceutically acceptable carrier.

Compositions, based on a unit dosage can comprise, from about 0.1 mg to about 1000 mg of sitaxentan or a pharmaceutically acceptable salt thereof, based on the weight of the sitaxentan active. Examples of other dosages are 1 mg, 10 mg, 50, mg, 100 mg, and 500 mg of sitaxentan or a pharmaceutically acceptable salt thereof, based on the weight of the sitaxentan active.

Compositions can also be prepared based on weight percentages.

In one embodiment the compositions useful here comprise from about 0.001% to about 25% by weight sitaxentan or a pharmaceutically salt thereof, based on the weight of the sitaxentan active.

In one embodiment the compositions useful here comprise from about 0.01% to about 10% by weight sitaxentan or a pharmaceutically salt thereof, based on the weight of the sitaxentan active.

In one embodiment the compositions useful here comprise from about 0.1% to about 5% by weight sitaxentan or a pharmaceutically salt thereof, based on the weight of the sitaxentan active.

In one embodiment the compositions useful here comprise from about 0.2% to about 3% by weight sitaxentan or a pharmaceutically salt thereof, based on the weight of the sitaxentan active.

For these foregoing compositions comprising a designated amount or weight percentage of the sitaxentan, the amount or weight percentage of the sitaxentan is determined or calculated based on the actual amount of the sitaxentan moiety, which has a molar mass of 454.906, and not including the additional weight provided by any counter ions when a sitaxentan salt is used. In other words, the compositions are based on the amount or weight percentage of the sitaxentan chemical moiety.

Furthermore, because the present invention is related to local or topical compositions and because it is highly desirable to limit systemic exposure, the unit dosage could be formulated to demonstrate at least one of the following pharmacokinetic parameters selected from a Cₘₐₓ less than about 13, μg/ml, or a Cₘₐₓ less than about 7 μg/ml or an AUC less than about 40 μg hr/ml. These pharmacokinetic parameters are based on those reported to the European Medicines Agency for Thelin.

Formulations for Topical Administration

In one embodiment, the compositions or formulations of the present invention comprise a selective ET-A receptor antagonist or pharmaceutically acceptable salt thereof and a pharmaceutically acceptable carrier. These formulations can be made using standard formulation and mixing techniques familiar to one of ordinary skill in the art of pharmaceuticals and formulations.

In one embodiment, the compositions or formulations of the present invention comprise sitaxentan or a pharmaceutically acceptable salt thereof and a pharmaceutically acceptable carrier. These formulations can be made using standard formulation and mixing techniques familiar to one of ordinary skill in the art of pharmaceuticals and formulations.

In one aspect, the pharmaceutical composition is selected from the group consisting of a gel, ointment, lotion, emulsion, cream, foam, mousse, liquid, paste, jelly, tape, spray, suspension, dispersion, or aerosol.

Useful herein are compositions wherein the pharmaceutically acceptable carrier is selected from one or more materials selected from sesame oil, mineral oil, olive oil, petrolatum, water, ethanol, ethanol/water mixtures, isopropanol, isopropanol/water mixtures, dimethyl sulfoxide, and dimethyl isosorbide. Other examples, but not all examples, of pharmaceutical carriers include those selected from oils derived from fruits or vegetables or flowers or nuts or seeds (including, but not limited to, sesame oil, peanut oil, and castor oil), alcohols (including, but not limited to, ethanol, benzyl alcohol, and isopropyl alcohol), dipropylene glycol, ethyl acetate, ethyl lactate, ethyl oleate, glycerin, isopropyl myristate, dimethyl sulfoxide, isopropyl palmitate, medium-chain triglycerides, mineral oil, polyethylene glycol, propylene glycol, tricaprylin, and water. A specific example of a pharmaceutically acceptable carrier is ethanol. These components can be employed and used at levels appropriate for the formulation based on the knowledge of one with ordinary skill in the pharmaceutical and formulation arts. The amounts could range from under 1 percent by weight to up 90 percent or even over 99 percent by weight.

Various additional ingredients can be used in the compositions of the present invention. The compositions can comprise one or more further ingredients selected from a penetration enhancer, a preservative, an antioxidant, an emulsifier, a surfactant or wetting agent, an emollient, a film-forming agent, or a viscosity modifying agent. These components can be employed and used at levels appropriate for the formulation based on the knowledge of one with ordinary skill in the pharmaceutical and formulation arts. The amounts could range from under 1 percent by weight to up to 90 percent or even over 99 percent by weight.

In one aspect, a penetration enhancer can be included. In another aspect, a preservative can be included. In another aspect, an antioxidant can be included. In another aspect, an emulsifier can be included. In another aspect, an emollient can be included. In another aspect, a viscosity modifying agent can be included. In another aspect, a surfactant or wetting agent can be included. In another aspect, a film forming agent can be included. In another aspect, the pharmaceutical composition is in the form selected from the group consisting of a gel, ointment, lotion, emulsion, cream, liquid, spray, suspension, jelly, foam, mousse, paste, tape, dispersion, aerosol. These components can be employed and used at levels appropriate for the formulation based on the knowledge of one with ordinary skill in the pharmaceutical and formulation arts.

In another aspect, the pharmaceutically acceptable carrier can comprise a material selected from the group consisting of alcohols (including but not limited to ethanol, benzyl alcohol, or isopropyl alcohol), acetone, albumin, oils derived from fruits or vegetables or flowers or nuts or seeds (including but not limited to almond oil, corn oil, cottonseed oil, coconut oil, sesame oil, olive oil, peanut oil, safflower oil, soybean oil, or sunflower oil), benzyl benzoate, butylene glycol, carbon dioxide, castor oil, dibutyl phthalate, diethyl phthalate, diethylene glycol, diethylene glycol monoethyl ether, dimethyl ether, dimethyl phthalate, dimethyl sulfoxide, dimethylacetamide, dipropylene glycol, ethyl acetate, ethyl lactate, ethyl oleate, glycerin, glyceryl monostearate, glycofurol, isopropyl myristate, isopropyl palmitate, light mineral oil, mineral oil, medium-chain triglycerides, methyl lactate, monoethanolamine, octyldodecanol, polyethylene glycol, polyoxyl 35 castor oil, propylene carbonate, propylene glycol, pyrrolidone, triacetin, tricaprylin, triethanolamine, triethyl citrate, triolein, and water, or a combination thereof. These components can be employed and used at levels appropriate for the formulation based on the knowledge of one with ordinary skill in the pharmaceutical and formulation arts. The amounts could range from under 1 percent by weight to up to 90 percent or even over 99 percent by weight.

In another aspect, the at least one penetration enhancer can be selected from the group consisting of alcohols (including but not limited to ethanol, benzyl alcohol, oleyl alcohol, or isopropyl alcohol), diethyl sebacate, diethylene glycol, dimethyl sulfoxide, glyceryl monooleate, glycofurol, isopropyl myristate, isopropyl palmitate, light mineral oil, lauric acid, linoleic acid, menthol, myristic acid, oleic acid, palmitic acid, polyoxyethylene alkyl ethers, polyoxyglycerides, propylene glycol, propylene glycol monolaurate, pyrrolidone, sodium lauryl sulfate, squalane, thymol, tricaprylin, triolein, and transcutol, or a combination thereof. These components can be employed and used at levels appropriate for the formulation based on the knowledge of one with ordinary skill in the pharmaceutical and formulation arts. The amounts could range from under 1 percent by weight to up to 90 percent by weight.

In another aspect, the at least one preservative can be selected from the group consisting of parabens (including butylparabens, ethylparabens, methylparabens, and propylparabens), acetone sodium bisulfite, alcohol, benzalkonium chloride, benzethonium chloride, benzoic acid, benzyl alcohol, boric acid, bronopol, butylated hydroxyanisole, butylene glycol, calcium acetate, calcium chloride, calcium lactate, cetrimide, cetylpyridinium chloride, chlorhexidine, chlorobutanol, chlorocresol, chloroxylenol, cresol, edetic acid, glycerin, hexetidine, imidurea, isopropyl alcohol, monothioglycerol, pentetic acid, phenol, phenoxyethanol, phenylethyl alcohol, phenylmercuric acetate, phenylmercuric borate, phenylmercuric nitrate, potassium benzoate, potassium metabisulfite, potassium nitrate, potassium sorbate, propionic acid, propyl gallate, propylene glycol, propylparaben sodium, sodium acetate, sodium benzoate, sodium borate, sodium lactate, sodium metabisulfite, sodium propionate, sodium sulfite, sorbic acid, sulfur dioxide, thimerosal, zinc oxide, and N-acetylcysteine, or a combination thereof. These components can be employed and used at levels appropriate for the formulation based on the knowledge of one with ordinary skill in the pharmaceutical and formulation arts. The amounts could range from under 1 percent by weight to up to 30 percent by weight.

In another aspect, the at least one antioxidant can be selected from the group consisting of acetone sodium bisulfite, alpha tocopherol, ascorbic acid, ascorbyl palmitate, butylated hydroxyanisole, butylated hydroxytoluene, citric acid monohydrate, dodecyl gallate, erythorbic acid, fumaric acid, malic acid, mannitol, sorbitol, monothioglycerol, octyl gallate, potassium metabisulfite, propionic acid, propyl gallate, sodium ascorbate, sodium formaldehyde sulfoxylate, sodium metabisulfite, sodium sulfite, sodium thiosulfate, sulfur dioxide, thymol, vitamin E polyethylene glycol succinate, and N-acetylcysteine, or a combination thereof. These components can be employed and used at levels appropriate for the formulation based on the knowledge of one with ordinary skill in the pharmaceutical and formulation arts. The amounts could range from under 1 percent by weight to up to 30 percent by weight.

In another aspect, the at least one emulsifier can be selected from the group consisting of acacia, agar, ammonium alginate, calcium alginate, carbomer, carboxymethylcellulose sodium, cetostearyl alcohol, cetyl alcohol, cholesterol, diethanolamine, glyceryl monooleate, glyceryl monostearate, hectorite, hydroxypropyl cellulose, hydroxypropyl starch, hypromellose, lanolin, lanolin alcohols, lauric acid, lecithin, linoleic acid, magnesium oxide, medium-chain triglycerides, methylcellulose, mineral oil, monoethanolamine, myristic acid, octyldodecanol, oleic acid, oleyl alcohol, palm oil, palmitic acid, pectin, phospholipids, poloxamer, polycarbophil, polyoxyethylene alkyl ethers, polyoxyethylene castor oil derivatives, polyoxyehtylene sorbitan fatty acid esters, polyoxyethylene stearates, polyoxyl 15 hydroxystearate, polyoxyglycerides, potassium alginate, propylene glycol alginate, propylene glycol dilaurate, propylene glycol monolaurate, saponite, sodium borate, sodium citrate dehydrate, sodium lactate, sodium lauryl sulfate, sodium stearate, sorbitan esters, starch, stearic acid, sucrose stearate, tragacanth, triethanolamine, tromethamine, vitamin E polyethylene glycol succinate, wax, and xanthan gum, or a combination thereof. These components can be employed and used at levels appropriate for the formulation based on the knowledge of one with ordinary skill in the pharmaceutical and formulation arts. The amounts could range from under 1 percent by weight to up to 30 percent by weight.

In another aspect, the at least one emollient can be selected from the group consisting of almond oil, aluminum monostearate, butyl stearate, canola oil, castor oil, cetostearyl alcohol, cetyl alcohol, cetyl palmitate, cholesterol, coconut oil, cyclomethicone, decyl oleate, diethyl sebacate, dimethicone, ethylene glycol stearates, glycerin, glyceryl monooleate, glyceryl monostearate, isopropyl isostearate, isopropyl myristate, isopropyl palmitate, lanolin, lanolin alcohols, lecithin, mineral oil, myristyl alcohol, octyldodecanol, oleyl alcohol, palm kernel oil, palm oil, petrolatum, polyoxyethylene sorbitan fatty acid esters, propylene glycol dilaurate, propylene glycol monolaurate, safflower oil, squalene, sunflower oil, tricaprylin, triolein, wax, xylitol, zinc acetate, or a combination thereof. These components can be employed and used at levels appropriate for the formulation based on the knowledge of one with ordinary skill in the pharmaceutical and formulation arts. The amounts could range from under 1 percent by weight to up to 60 percent by weight.

In another aspect, the at least one viscosity modifying agent can be selected from the group consisting of acacia, agar, alginic acid, aluminum monostearate, ammonium alginate, attapulgite, bentonite, calcium alginate, calcium lactate, carbomer, carboxymethylcellulose calcium, carboxymethylcellulose sodium, carrageenan, cellulose, ceratonia, ceresin, cetostearyl alcohol, cetyl palmitate, chitosan, colloidal silicon dioxide, corn syrup solids, cyclomethicone, ethylcellulose, gelatin, glyceryl behenate, guar gum, hectorite, hydrophobic colloidal silica, hydroxyethyl cellulose, hydroxyethylmethyl cellulose, hydroxypropyl cellulose, hydroxypropyl starch, hypromellose, magnesium aluminum silicate, maltodextrin, methylcellulose, myristyl alcohol, octyldodecanol, palm oil, pectin, polycarbophil, polydextrose, polyethylene oxide, polyoxyethylene alkyl ethers, polyvinyl alcohol, potassium alginate, propylene glycol alginate, pullulan, saponite, sodium alginate, starch, sucrose, sugar, sulfoburylether μ-cyclodextrin, tragacanth, trehalose, and xanthan gum, or a combination thereof. These components can be employed and used at levels appropriate for the formulation based on the knowledge of one with ordinary skill in the pharmaceutical and formulation arts. The amounts could range from under 1 percent by weight to up to 60 percent.

In another aspect, the at least one film forming agent can be selected from the group consisting of ammonium alginate, chitosan, colophony, copovidone, ethylene glycol and vinyl alcohol grafted copolymer, gelatin, hydroxypropyl cellulose, hypromellose, hypromellose acetate succinate, polymethacrylates, poly(methyl vinyl ether/maleic anhydride), polyvinyl acetate dispersion, polyvinyl acetate phthalate, polyvinyl alcohol, povidone, pullulan, pyroxylin, and shellac, or a combination thereof. These components can be employed and used at levels appropriate for the formulation based on the knowledge of one with ordinary skill in the pharmaceutical and formulation arts. The amounts could range from under 1 percent by weight to up to 90 percent or even over 99 percent by weight.

In another aspect, the at least one surfactant or wetting agent can be selected from the group consisting of docusate sodium, phospholipids, sodium lauryl sulfate, benzalkonium chloride, cetrimide, cetylpyridinium chloride, alpha tocopherol, glyceryl monooleate, myristyl alcohol, poloxamer, polyoxyethylene alkyl ethers, polyoxyethylene castor oil derivatives, polyoxyethylene sorbitan fatty acid esters, polyoxyethylene stearates, polyoxyl 15 hydroxystearate, polyoxyglycerides, propylene glycol dilaurate, propylene glycol monolaurate, sorbitan esters, sucrose stearate, tricaprylin, and vitamin E polyethylene glycol succinate, or a combination thereof. These components can be employed and used at levels appropriate for the formulation based on the knowledge of one with ordinary skill in the pharmaceutical and formulation arts. The amounts could range from under 1 percent by weight to up to 30 percent by weight.

In another aspect, a buffering agent can be included. In another aspect, an emollient can be included. In another aspect, an emulsifying agent can be included. In another aspect, an emulsion stabilizing agent can be included. In another aspect, a gelling agent can be included. In another aspect, a humectant can be included. In another aspect, an ointment base or oleaginous vehicle can be included. In another aspect, a suspending agent can be included. In another aspect an acidulant can be included. In another aspect, an alkalizing agent can be included. In another aspect, a bioadhesive material can be included. In another aspect, a colorant can be included. In another aspect, a microencapsulating agent can be included. In another aspect, a stiffening agent can be included. These components can be employed and used at levels appropriate for the formulation based on the knowledge of one with ordinary skill in the pharmaceutical and formulation arts. The amounts could range from under 1 percent by weight to up to 90 percent or even over 99 by weight.

One of ordinary skill in the pharmaceutical and formulation arts can determine the appropriate levels of the essential and optional components of the compositions of the present invention.

Methods of preparing the sitaxentan compositions are also intended as part of the present invention and would be apparent to one of ordinary skill in the pharmaceutical and formulation arts using standard formulation and mixing techniques.

Methods of Treatment

The present invention utilizes a therapeutically effective amount of sitaxentan or a pharmaceutically acceptable salt thereof and a pharmaceutically acceptable carrier for providing local or topical compositions for treating conditions such as cutaneous fibrosis and connective tissue diseases. Such conditions can include scleroderma (including, but not limited to, systemic sclerosis and localized scleroderma), Raynaud's phenomenon, Peyronie's disease, sclerodactyly, cutaneous ulcers, morphea, en coup de sabre, cicatricial alopecia, scarring alopecia (including, but not limited to, lichen planopilaris, frontal fibrosing alopecia, central centrifugal cicatricial alopecia, folliculitis decalvens, discoid lupus erythematous, and dissecting cellulitis), rheumatoid arthritis, lupus, lichen sclerosis, keloid scars, hypertrophic scars, burn scars, and combinations thereof.

The methods comprise locally or topically applying a therapeutically effective amount of sitaxentan, or a pharmaceutically acceptable salt thereof, to the mammal, such as a human patient, in need thereof. When a human patient is being treated, the composition is applied to the skin of said human.

Various dosing regimens can be prescribed and used based on the skill and knowledge of the physician or other practitioner. In some embodiments, a unit dosage of the composition, as described herein can be applied at least once daily. In other embodiments, a unit dosage of the composition can be applied at least twice daily, or at least once weekly, or at least twice weekly.

Local or topical administration of the composition can be continued in the judgment of the physician or practitioner until the desired therapeutic benefit is achieved, i.e. until the cutaneous fibrosis or the connective tissue disease is treated. In some instances, it can be desirable to continue long term or chronic therapy.

### EXAMPLES

The following examples further describe and demonstrate embodiments within the scope of the present invention. The Examples are given solely for purpose of illustration and are not to be construed as limitations of the present invention, as many variations thereof are possible without departing from the spirit and scope of the invention.

### Example 1

### Effect of Sitaxentan on TGF-β1 Induced Fibroblasts in Male Cells

The effect of sitaxentan in a wound closure assay was measured using male normal human dermal fibroblasts induced with TGF-β1 into a profibrotic phenotype. Scleroderma fibroblasts “close” the wound in a scratch assay significantly faster than controls, and these induced fibroblasts behave similarly to scleroderma fibroblasts in a 2-dimensional scratch assay. For this assay the cells were grown to confluence, a scratch/ablation, i.e. “wound”, was created, and migration across the cleared zone was tracked. See, http://journals.plos.org/plosone/article?id=10.1371/journal.pone.0007438 and Wu, M. et al. Rosiglitazone abrogates bleomycin-induced scleroderma and blocks profibrotic responses through peroxisome proliferator-activated receptor-gamma. Am J Pathol174, 519-533, doi:10.2353/ajpath.2009.080574 (2009)

Male normal human dermal fibroblast cells (LLCT FC0024 lot 03869_male fibroblast, 23 year old) were seeded to confluence in 96 well plates in 10% fetal bovine serum (FBS) Dulbecco's modified eagle medium (DMEM).

The cells were washed to remove the FBS, and serum free media was added for 16 hr overnight (O/N).

Scratch assays were performed across each confluent monolayer.

The samples were optionally stained with CellTracker Green (5 uM) to produce fluorescence time zero images.

The cells were treated with increasing concentrations (1 μM, 3 μM, 10 μM, 30 μM, and 100 μM) of vehicle control, sitaxentan, and bosentan (as a comparator compound), in the presence of 50 ng/mL TGF-β1 to induce fibrogenesis. Six replicate samples were run for each concentration.

Time zero images were taken and initial distance of the scratch/ablation recorded.

The samples were incubated for 24 hours at 37° C., after which the media was removed and 0.5 pg/mL calcein acetoxymethyl (calcein AM) was added.

The samples were incubated for 30 minutes.

Images were taken and the distance of the scratch/ablation recorded.

Decreases in distance indicated “wound” closure.

Distances were calculated and analyzed using GraphPad Prism 7.

The data are presented in Table 1 as distance changes in pm (micrometers).

TABLE 1
Scratch Assay Results

| | Concentration of Test Material | | | | |
|---|---|---|---|---|---|
| | 1 μM | 3 μM | 10 μM | 30 μM | 100 μM |
| VC | 569.1 | 724.8 | 660.7 | 754.0 | 588.7 |
| SIT | 505.8 | 557.0 | 496.0 | 493.9 | 268.6 |
| BOS | 478.3 | 356.0 | 359.2 | 534.7 | 413.7 |

VC = vehicle control,
SIT = sitaxentan,
BOS = bosentan

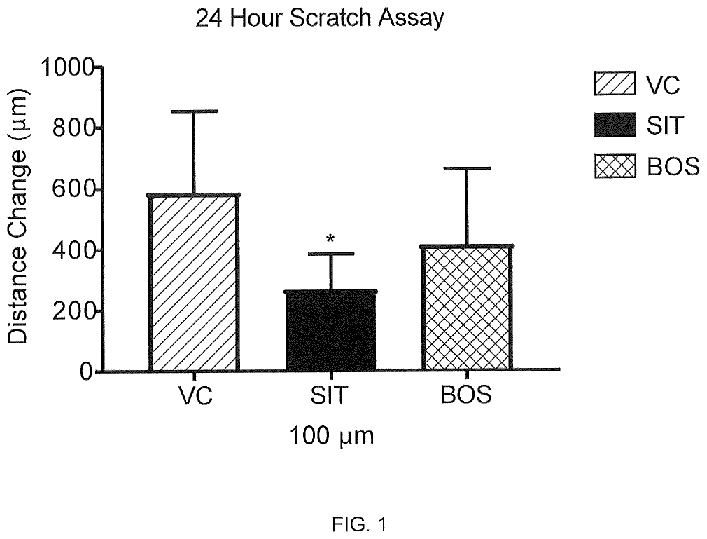

The data for the 100 μM concentrations of vehicle control, sitaxentan, and bosentan are presented as bar graphs with statistical analyses in FIG. 1.

These results show that sitaxentan significantly reduced the migration of TGF-β1 induced male normal human dermal fibroblasts, whereas bosentan had no significant effect.

### Example 2

### Effect of Sitaxentan on TGF-β1 Induced Fibroblasts in Female Cells

The effect of sitaxentan in a wound closure assay was measured using female normal human dermal fibroblasts induced with TGF-β1 into a profibrotic phenotype. Scleroderma fibroblasts “close” the wound in a scratch assay significantly faster than controls, and these induced fibroblasts behave similarly to scleroderma fibroblasts in a 2-dimensional scratch assay. For this assay the cells were grown to confluence, a scratch/ablation, i.e. “wound”, was created, and migration across the cleared zone was tracked. See, http://journals.plos.org/plosone/article?id=10.1371/journal.pone.0007438 and Wu, M. et al. Rosiglitazone abrogates bleomycin-induced scleroderma and blocks profibrotic responses through peroxisome proliferator-activated receptor-gamma. Am J Pathol174, 519-533, doi:10.2353/ajpath.2009.080574 (2009)

Female normal human dermal fibroblast cells (LLCT FC0024 lot 00703_female fibroblast, 45 year old) were seeded to confluence in 96 well plates in 10% fetal bovine serum (FBS) Dulbecco's modified eagle medium (DMEM).

The cells were washed to remove the FBS, and serum free media was added for 16 hr overnight (O/N).

Scratch assays were performed across each confluent monolayer.

The samples were optionally stained with CellTracker Green (5 uM) to produce fluorescence time zero images.

The cells were treated with increasing concentrations (3 μM, 10 μM, 30 μM, and 100 μM) of vehicle control, sitaxentan, and bosentan (as a comparator compound), in the presence of 50 ng/mL TGF-β1 to induce fibrogenesis. Three replicate samples were run for each concentration.

Time zero images were taken and initial distance of the scratch/ablation recorded.

The samples were incubated for 48 hours at 37° C., after which the media was removed and 0.5 μg/mL calcein acetoxymethyl (calcein AM) was added.

The samples were incubated for 30 minutes.

Images were taken and the distance of the scratch/ablation recorded.

Decreases in distance indicated “wound” closure.

Distances were calculated and analyzed using GraphPad Prism 7.

The data are presented in Table 2 as distance changes in μm (micrometers).

TABLE 2
Scratch Assay Results

Concentration of Test Material

| | 3 μM | 10 μM | 30 μM | 100 μM |
|---|---|---|---|---|
| VC | 673.1 | 847.7 | 713.6 | 775.0 |
| SIT | 554.0 | 560.4 | 331.1 | −55.1 |
| BOS | 510.4 | 564.4 | 456.0 | 170.0 |

VC = vehicle control,
SIT = sitaxentan,
BOS = bosentan

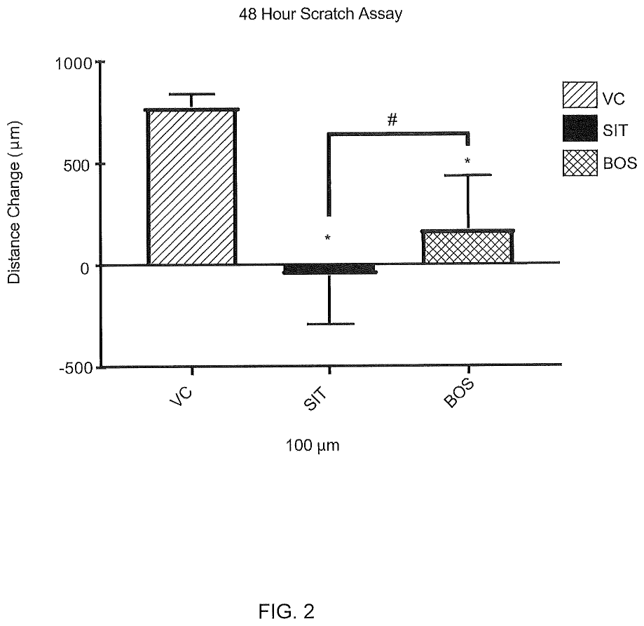

The data for the 100 μM concentrations of vehicle control, sitaxentan, and bosentan are presented as bar graphs with statistical analyses in FIG. 2.

These results show that both sitaxentan and bosentan significantly reduced the migration of TGF-β1 induced female normal human dermal fibroblasts compared to the vehicle control, with sitaxentan reducing migration significantly more than bosentan.

### Example 3

### Effect of Sitaxentan on Collagen Production in TGF-β1 Induced Human Dermal Fibroblasts

The effect of sitaxentan on collagen production was measured in an AlphaLISA assay using male normal human dermal fibroblasts induced with TGF-β1 into a profibrotic phenotype. For this assay cells were grown for 48 hours in the presence of vehicle control, sitaxentan, and bosentan. See, http://www.perkinelmer.com/product/alphalisa-hpip-collagen-kit-100pts-al353hv.

An AlphaLISA assay was used, which is a variation of FRET (Fluorescence resonance energy transfer) technology that allows for the detection of molecules of interest in a no-wash, highly sensitive, quantitative assay. In an AlphaLISA assay, a biotinylated anti-analyte antibody binds to Streptavidin-coated donor beads while another anti-analyte antibody is conjugated to AlphaLISA Acceptor beads. In the presence of the analyte, the beads come into close proximity. The excitation of the donor beads cause the release of singlet oxygen molecules that trigger a cascade of energy transfer in the acceptor beads, resulting in a sharp peak of light emission at 615 nm.

Male normal human dermal fibroblast cells (LLCT FC0024 lot 03869_male fibroblast, 23 year old) were seeded to confluence in 96 well plates in 10% fetal bovine serum (FBS) Dulbecco's modified eagle medium (DMEM).

The cells were washed to remove the FBS, and serum free media was added overnight (O/N).

The cells were then stimulated with 50 ng/mL TGF-β1 and treated.

The supernatant media above the cells in the wells was collected and diluted 1:20 in serum-free DMEM media.

5 μL of each hPIP analyte standard or 5 μL of sample was added.

10 μL of 5× AlphaLlSA Anti-hPIP Acceptor beads was added (10 μg/mL final).

The plate was incubated 30 minutes at 23° C.

10 μL of 5× Biotinylated Anti-hPIP Antibody was added (1 nM).

The plate was incubated 60 minutes at 23° C.

25 μL of 2× Streptavidin-Donor beads were added (40 μg/mL final).

The plate was incubated 30 minutes at 23° C. in the dark.

The plate was read using a Perkin Elmer EnVision-Alpha Reader (615 nm).

Data were analyzed using GraphPad Prism 7.

Three to four replicates were run for each sample.

The data are presented in Table 3 as the human procollagen Type I C-peptide (HPIP) level in ng/mL (nanograms/mL).

TABLE 3
Collagen Level Results

| | Concentration of Test Material | | | | |
|---|---|---|---|---|---|
| | 1 μM | 3 μM | 10 μM | 30 μM | 100 μM |
| VC | 7.9 | 8.1 | 7.7 | 8.6 | 9.6 |
| SIT | 7.0 | 7.1 | 6.4 | 5.8 | 2.2 |
| BOS | 8.2 | 8.6 | 7.9 | 7.1 | 6.8 |
| VC-NT | — | — | — | — | 4.0 |

VC = vehicle control,
SIT = sitaxentan,
BOS = bosentan
VC-NT = vehicle control with no TGF-β1 treatment

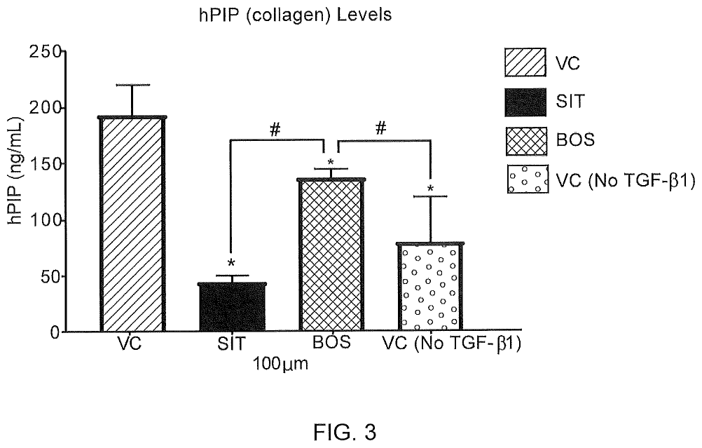

The data for the 100 μM concentrations of vehicle control, sitaxentan, and bosentan are presented as bar graphs with statistical analyses in FIG. 3.

These results show that both sitaxentan and bosentan significantly decreased elevated collagen levels in TGF-β1 induced male normal human dermal fibroblasts compared to the vehicle control, with sitaxentan being significantly more efficacious than bosentan and returning collagen levels to untreated / uninduced levels.

### Example 4

### Effect of Sitaxentan on Cell Viability, Cell Cytotoxicity, and Apoptosis in TGF-β1 Induced Human Dermal Fibroblasts

The effect of sitaxentan on cell viability, cell cytotoxicity, and apoptosis was measured in an assay using male normal human dermal fibroblasts induced with TGF-β1 into a profibrotic phenotype. For these assays cells were grown for 48 hours in the presence of vehicle control, sitaxentan, and bosentan. The appropriate assay reagents and measuring techniques were used as indicated herein.

Male normal human dermal fibroblast cells (LLCT FC0024 lot 03869_male fibroblast, 23 year old) were seeded to confluence in 96 well plates in 10% fetal bovine serum (FBS) Dulbecco's modified eagle medium (DMEM).

The cells were washed to remove the FBS, and serum free media is added overnight (O/N).

The cells were then stimulated with 50 ng/mL TGF-β1 and treated for 48 hours.

The following reagents were used for the different assays:

For the cell viability/cytotoxicity assay:

A master mix of the viability/cytotoxicity reagent was made by combining 10 uL of each substrate (GF-AFC and bis-AAF-R110) to 2 mLs of Assay Buffer (Promega, Cat #G6320). 20 μl of this viability/cytotoxicity reagent was then added to each well and briefly mixed. The plate was incubated for 30 minutes at 37° C. prior to measuring fluorescence at: 400Ex/505Em (Viability) and 485Ex/520Em (Cytotoxicity).

For the apoptosis assay:

100 μl of Caspase-Glo® 3/7 Reagent (Promega, Cat #G6320), for the apoptosis assay, was subsequently added after the viability/cytotoxicity fluorescent reads and briefly mixed.

The plate was incubated for an additional 30 minutes at room temperature prior to the measurement of luminescence to detect apoptosis.

Data were analyzed using GraphPad Prism 7.

Six to nine replicates were run for each sample.

For cell viability:

The data are presented in Table 4A as the relative fluorescence units (RFUs) as a measure of cell viability.

TABLE 4A
Cell Viability

| | Concentration of Test Material | | | | |
|---|---|---|---|---|---|
| | 1 μM | 3 μM | 10 μM | 30 μM | 100 μM |
| VC | 5528.7 | 5516.3 | 5560.2 | 5400.3 | 5353.7 |
| SIT | 5678.3 | 5639.0 | 5600.5 | 5250.8 | 5171.0 |
| BOS | 5406.8 | 5548.5 | 5521.2 | 5537.0 | 5584.3 |

VC = vehicle control,
SIT = sitaxentan,
BOS = bosentan

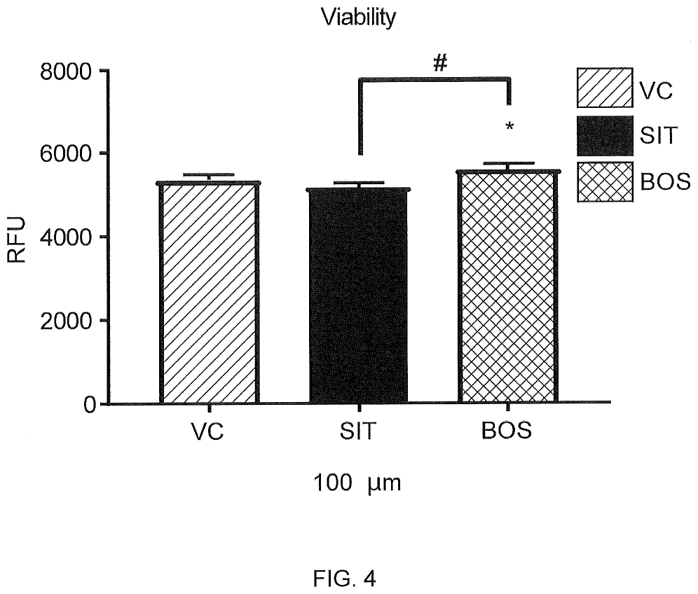

The data from Table 4A for the 100 μM concentrations of vehicle control, sitaxentan, and bosentan are presented as bar graphs with statistical analyses in FIG. 4.

These results show that no significant toxicity was observed for sitaxentan in TGF-β1 induced male normal human dermal fibroblasts at concentrations up to 100 μM.

For cell cytotoxicity:

The data are presented in Table 4B as the relative fluorescence units (RFUs) as a measure of cell cytotoxicity.

TABLE 4B
Cell Cytotoxicity

| | Concentration of Test Material | | | | |
|---|---|---|---|---|---|
| | 1 μM | 3 μM | 10 μM | 30 μM | 100 μM |
| VC | 3837.5 | 3801.2 | 3844.7 | 3496.3 | 3396.8 |
| SIT | 4643.9 | 5058.3 | 4741.7 | 3837.5 | 2911.3 |
| BOS | 4256.9 | 3954.2 | 3764.0 | 4607.5 | 3929.0 |

VC = vehicle control,
SIT = sitaxentan,
BOS = bosentan

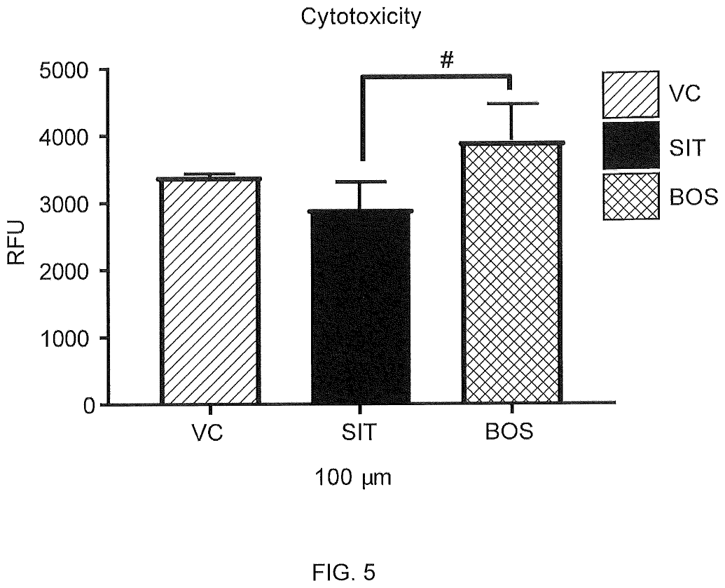

The data from Table 4B for the 100 μM concentrations of vehicle control, sitaxentan, and bosentan are presented as bar graphs with statistical analyses in FIG. 5.

These results show that neither bosentan nor sitaxentan was significantly more cytotoxic than the vehicle control; however, bosentan was significantly more cytotoxic than sitaxentan in TGF-β1 induced male normal human dermal fibroblasts.

For apoptosis:

The data are presented in Table 4C as the relative light units (RLUs) as a measure of cell apoptosis.

TABLE 4C
Apoptosis

| | Concentration of Test Material | | | | |
|---|---|---|---|---|---|
| | 1 μM | 3 μM | 10 μM | 30 μM | 100 μM |
| VC | 36833.3 | 40552.8 | 37338.7 | 37284.0 | 35834.2 |
| SIT | 37593.4 | 39162.8 | 39512.0 | 46053.5 | 51050.2 |
| BOS | 39366.8 | 38639.8 | 38586.7 | 42825.2 | 43330.7 |

VC = vehicle control,
SIT = sitaxentan,
BOS = bosentan

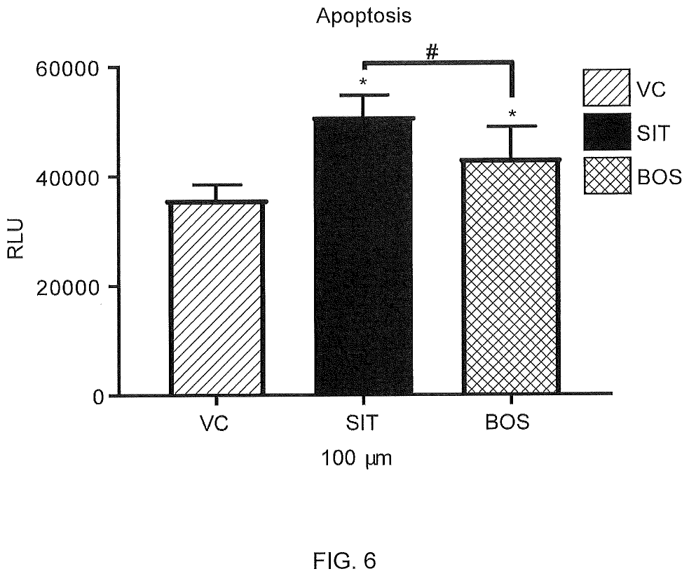

The data from Table 4C for the 100 μM concentrations of vehicle control, sitaxentan, and bosentan are presented as bar graphs with statistical analyses in FIG. 6.

These results show that apoptosis of TGF-β1 induced male normal human dermal fibroblasts was elevated after treatment with both sitaxentan and bosentan, but with sitaxentan having a significantly more potent effect than bosentan.

### Example 5

### Preparation of a Composition for Topical Delivery

Sitaxentan sodium is mixed with ethanol to provide a 1% solution based on the weight of the sitaxentan active.

This composition is useful for topical administration to a human patient or animal for the treatment of conditions such as cutaneous fibrosis or a connective tissue disease.

Incorporation by Reference

The entire disclosure of each of the patent documents, including certificates of correction, patent application documents, scientific articles, governmental reports, websites, and other references referred to herein is incorporated by reference herein in its entirety for all purposes. In case of a conflict in terminology, the present specification controls.

Equivalents

The invention can be embodied in other specific forms without departing from the spirit or essential characteristics thereof. The foregoing embodiments are to be considered in all respects illustrative rather than limiting on the invention described herein. In the various embodiments of the methods and systems of the present invention, where the term comprises is used with respect to the recited steps of the methods or components of the compositions, it is also contemplated that the methods and compositions consist essentially of, or consist of, the recited steps or components. Furthermore, it should be understood that the order of steps or order for performing certain actions is immaterial so long as the invention remains operable. Moreover, two or more steps or actions can be conducted simultaneously.

In the specification, the singular forms also include the plural forms, unless the context clearly dictates otherwise. Unless defined otherwise, all technical and scientific terms used herein have the same meaning as commonly understood by one of ordinary skill in the art to which this invention belongs. In the case of conflict, the present specification will control.

Furthermore, it should be recognized that in certain instances a composition can be described as being composed of the components prior to mixing, because upon mixing certain components can further react or be transformed into additional materials.

All percentages and ratios used herein, unless otherwise indicated, are by weight.

1. A method for treating cutaneous fibrosis or a connective tissue disease, comprising locally or topically applying a therapeutically effective amount of a selective endothelin-A (ET-A) receptor antagonist or inhibitor to a mammal in need thereof.

2. A method according to claim 1 wherein the selective endothelin-A (ET-A) receptor antagonist or inhibitor has a selectivity of at least two-fold over endothelin-B (ET-B), or a selectivity of at least five-fold over endothelin-B (ET-B)), or a selectivity of at least ten-fold over endothelin-B (ET-B)), or a selectivity of at least 100-fold over endothelin-B (ET-B)), or a selectivity of at least 1000-fold over endothelin-B (ET-B)), or a selectivity of at least 5000-fold over endothelin-B (ET-B).

3. (canceled)

4. (canceled)

5. (canceled)

6. (canceled)

7. (canceled)

8. A method according to claim 1 wherein the selective endothelin-A antagonist or inhibitor is sitaxentan or a pharmaceutically acceptable salt thereof.

9. A method according to claim 1 wherein the mammal is a human patient.

10. A method according to claim 1 wherein the cutaneous fibrosis or connective tissue disease is selected from scleroderma, systemic sclerosis, localized scleroderma, diffuse systemic sclerosis, limited systemic sclerosis, Raynaud's phenomenon, Peyronie's disease, sclerodactyly, cutaneous ulcers, morphea, en coup de sabre, cicatricial alopecia, scarring alopecia, lichen planopilaris, frontal fibrosing alopecia, central centrifugal cicatricial alopecia, folliculitis decalvans, discoid lupus erythematous, dissecting cellulitis, rheumatoid arthritis, lupus, lichen sclerosis, keloid scars, hypertrophic scars, burn scars, and combinations thereof.

11. A method according to claim 8 wherein the pharmaceutically acceptable salt is selected from an alkali metal salt, an alkaline earth metal salt, and an ammonium salt.

12. A method according to claim 11 wherein the alkali metal salt is selected from lithium, sodium, and potassium.

13. A method according to claim 11 wherein the alkali metal salt is sodium.

14. (canceled)

15. A method according to claim 1 wherein the selective endothelin-A (ET-A) receptor antagonist or inhibitor is applied at least one daily.

16. A method according to claim 1 wherein the selective endothelin-A (ET-A) receptor antagonist or inhibitor is applied at least twice daily.

17. A method according to claim 1 wherein the selective endothelin-A (ET-A) receptor antagonist or inhibitor is applied at least once weekly.

18. A method according to claim 1 wherein the selective endothelin-A (ET-A) receptor antagonist or inhibitor is applied at least twice weekly.

19. A method according to claim 1 wherein the selective endothelin-A (ET-A) receptor antagonist or inhibitor is applied at least once daily until the cutaneous fibrosis or connective tissue disease is treated.

20. A method according to claim 1 wherein the selective endothelin-A (ET-A) receptor antagonist or inhibitor is applied from a pharmaceutically acceptable composition.

21. A method according to claim 1 for treating cutaneous fibrosis or a connective tissue disease, comprising locally or topically applying a pharmaceutically acceptable composition comprising a therapeutically effective amount of sitaxentan or a pharmaceutically acceptable salt thereof.

22. (canceled)

23. (canceled)

24. (canceled)

25. (canceled)

26. (canceled)

27. (canceled)

28. (canceled)

29. A method according to claim 21 wherein the composition is in the form of a unit dosage composition.

30. A method according to claim 29 wherein the unit dosage comprises from about 0.01 to about 1000 mg of sitaxentan or a pharmaceutically acceptable salt thereof, based on the weight of the sitaxentan active.

31. A method according to claim 29 wherein the unit dosage comprises from about 0.001% to about 25% by weight sitaxentan or a pharmaceutically acceptable salt thereof, based on the weight of the sitaxentan active, or from about 0.01% to about 10% by weight sitaxentan or a pharmaceutically acceptable salt thereof, based on the weight of the sitaxentan active, or from about 0.1% A to about 5% by weight sitaxentan or a pharmaceutically acceptable salt thereof, based on the weight of the sitaxentan active, or from about 0.2% to about 3% by weight sitaxentan or a pharmaceutically acceptable salt thereof, based on the weight of the sitaxentan active.

32. (canceled)

33. (canceled)

34. (canceled)

35. A method according to claim 29 wherein the unit dosage demonstrates at least one of the following pharmacokinetic parameters selected from a Cₘₐₓ less than about 13 μg/ml, or a Cₘₐₓ less than about 7 μg/ml, or an AUC less than about 40 μg hr/m I.

36. (canceled)

37. A composition for local or topical delivery comprising a therapeutically effective amount of a selective endothelin-A (ET-A) receptor antagonist or inhibitor and a pharmaceutically acceptable carrier.

38. A composition according to claim 37 wherein the selective endothelin-A antagonist or inhibitor is sitaxentan or a pharmaceutically acceptable salt thereof.
